(19) United States
(12) Patent Application Publication
(10) Pub. No.: US 2010/0232568 A1
(43) Pub. Date: Sep. 16, 2010

(54) X-RAY OR GAMMA RAY DETECTOR ARRAY

(76) Inventors: Björn HEISMANN, Erlangen (DE); Jürgen LEPPERT, Forchheim (DE)

Correspondence Address: HARNESS, DICKEY & PIERCE, P.L.C., P.O.BOX 8910, RESTON, VA, 20195, US

(21) Appl. No.: 12/722,636

(22) Filed: Mar. 12, 2010

(30) Foreign Application Priority Data
Mar. 16, 2009 (DE) 10 2009 013 301.1

(51) Int. Cl. A61B 6/03; G01T 1/20; G01T 1/24; G01T 1/161; G01T 1/166

(52) U.S. Cl. 378/19; 250/367; 250/370.09; 250/394

(57) ABSTRACT

An X-ray or gamma ray detector array is disclosed, with at least one row of detector elements including at least one layer made of an X-ray or gamma radiation absorbing conversion material. In at least one embodiment, the conversion material is at least in part composed of a chemical element with an atomic number ≧72, preferably >82. Such an X-ray or gamma ray detector array allows an increased spatial and spectral resolution and better dose usage.

### PRIORITY STATEMENT

The present application hereby claims priority under 35 U.S.C. §119 on German patent application number DE 10 2009 013 301.1 filed Mar. 16, 2009, the entire contents of which are hereby incorporated herein by reference.

### FIELD

At least one embodiment of the present invention generally relates to an X-ray or gamma ray detector array with at least one row of detector elements having at least one layer made of an X-ray or gamma radiation absorbing conversion material.

### BACKGROUND

By way of example, X-ray or gamma ray detector arrays are used in medical imaging in order to obtain images of the interior of the body of a patient. X-ray or gamma ray detector arrays can also be used in the field of security technology, for example for X-raying baggage, and in other technical fields.

In general, CT equipment with scintillator detectors are currently used in computed tomography (CT). The detector elements of a scintillator detector have a conversion material layer, which absorbs X-ray radiation and converts the absorbed X-ray radiation into optical radiation, which then can be detected by appropriate photodetectors below the layer of the conversion material. Known conversion materials for scintillator detectors are GdOS, YGO or LuTAG. Additionally, detectors with Cd(Zn)Te semiconductor materials are currently being developed. These semiconductor materials can directly convert the absorbed X-ray radiation into electrical signals. Thus, this type of detector is also referred to as a direct convertor.

However, the problem of so-called fluorescence crosstalk occurs in both the previously known direct convertors and in the known scintillator detectors. This is understood to mean that, during the absorption of an X-ray quantum, part of the X-ray energy, as a secondary quantum, can reach regions of the conversion material layer that are situated further away and is therefore detected by another detector element of the detector array. Here, the secondary X-ray quantum has a significant average range, which is e.g. approximately 300 to 400 μm in GdOS. It is also possible that the secondary quantum leaves the detector and it is not detected in this case.

The fluorescence crosstalk reduces the spatial and spectral resolution of the detector array. This effect also impairs the dose usage because there is an energy loss as a result of the energy transfer to adjacent detector elements or the loss of the secondary quantum.

### SUMMARY

In at least one embodiment of the present invention, an X-ray or gamma ray detector array is provided with an increased spatial resolution, in which the problem of fluorescence crosstalk does not occur, or at least only occurs with a reduced strength.

Advantageous refinements of the detector array are the subject matter of the dependent patent claims or emerge from the following description and the example embodiment.

The proposed X-ray or gamma ray detector array comprises at least one row of detector elements having at least one layer made of an X-ray or gamma radiation absorbing conversion material. The detector array is distinguished by the fact that the conversion material is at least in part composed of a chemical element with an atomic number ≧72, preferably >82.

The chemical element with this high atomic number forms the main component of the conversion material for absorbing the X-ray or gamma radiation. As a result of the high atomic number, this material has an upper K-edge with an energy that is higher than the energies of the K-edges of the chemical elements previously used as the conversion material. The high K-edge significantly reduces the amount of fluorescence crosstalk. This can be further optimized in X-ray systems by matching the acceleration voltage of the X-ray tube to the position of the K-edge. Here, the acceleration voltage of the X-ray tube is set to generate X-ray radiation with a maximum energy lying below the K-edge of this chemical element with the high atomic number. In computed tomography, typical acceleration voltages of X-ray tubes lie in the region of between 80 and 140 kV. Operation of these X-ray tubes at acceleration voltages in the lower range, particularly at 90 kV, therefore leads to a further reduction in the fluorescence crosstalk in the aforementioned conversion material, the upper K-edge energies of which lie above 80 keV.

The use of such a conversion material in an X-ray or gamma ray detector array thus leads to an increased spatial resolution due to the reduced fluorescence crosstalk and this also allows a reduction in the detection surfaces of the detector elements, also referred to as the pixel size, especially in the case of counting detectors. Here, sizes of the detection surfaces with dimensions of ≦250 μm in both dimensions are possible. This conversion material also improves the dose usage of the detectors and, in the case of counting detectors, the energy resolution as well.

In a preferred refinement, the conversion material is composed of a combination of at least two different chemical elements. Here, the first chemical element has an atomic number of ≧72, or of >82, while the second chemical element has an atomic number of ≦53, preferably of ≦16. Herein, the K-edge of the lighter chemical element is ≦30 keV (K-edge of iodine) and thus it is in the region of the low-energetic end of the emitted X-ray energy spectrum in CT equipment. If the X-ray energy spectrum, the energetic position of which can be adjusted by the acceleration voltage in the X-ray tube, is in the energy range between the two K-edges of the chemical elements used in the conversion material, for example between and 90 keV, fluorescence crosstalk can be avoided almost completely. Even at higher energies, for example of up to 140 keV in computed tomography or in the case of SPECT tracers, this results in a further improvement in the spatial resolution compared to the previously known X-ray or gamma ray detector arrays.

The conversion material is preferably formed from a combination of one or more of the chemical elements W, Tl, Hg, Pb or Bi with the chemical elements O, S, Se, Te or I. X-ray or gamma ray detector arrays with these element combinations can be implemented without problems and offer the aforementioned advantages.

The proposed detector array can be designed as both a direct convertor, if suitable semiconductor materials are used, and a scintillator detector. It can be either an integrating detector or a counting detector. The advantages offered by counting detectors are those of negligible electronics noise, a good contrast-to-noise ratio and an intrinsically available energy resolution. A person skilled in the art is aware of the differences in the electronic design of counting and integrating detectors because such detectors are part of the prior art.

The proposed X-ray or gamma ray detector array can be used in all technical fields in which X-ray or gamma radiation is intended to be detected in a spatially resolved fashion. By way of example, such a detector array can be used as a combined detector in PET/CT (PET: positron emission tomography) or in SPECT/CT (SPECT: single photon emission computed tomography). Such a detector array also can be used as a direct convertor for PET itself. The absorbability of the heavy chemical elements used in the conversion material is significantly above that of CdTe and so the detectors can be designed with a thinner conversion layer and thus, possibly, in a more cost-effective fashion as well. There is no fluorescence crosstalk in the detector up to the K-edge of the heavy chemical element, e.g. 90 keV for Bi. This significantly increases the spatial and spectral resolution of the detector array.

### BRIEF DESCRIPTION OF THE DRAWINGS

In the following text, the proposed X-ray or gamma ray detector array will once again be explained briefly on the basis of an example embodiment in conjunction with the drawings, in which

FIG. 1 shows an example of the design of a CT system with the proposed detector array; and

FIG. 2 shows a cross section of an example of the design of the proposed detector array.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

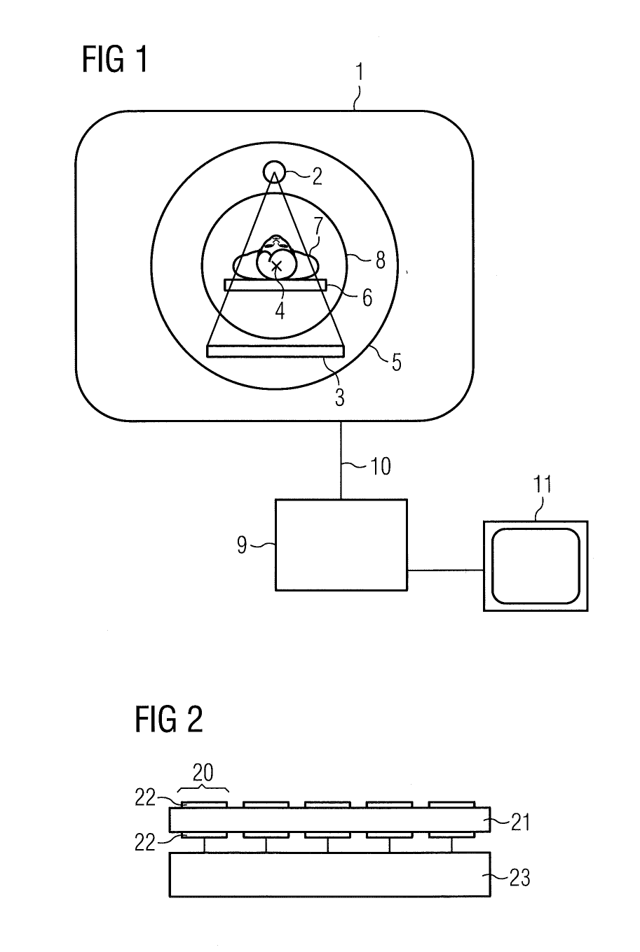

FIG. 1 shows a schematic illustration of a computed tomography scanner 1 into which a detector array according to the present invention can be inserted. In a conventional fashion, the computed tomography scanner 1 has a scanning system, which comprises a rotating frame 5 with an X-ray tube 2 attached thereto and the X-ray detector array 3 situated opposite the X-ray tube. A recording opening 8 is provided between the X-ray tube 2 and the X-ray detector array 3, into which opening a patient 7, lying on a moveable patient couch 6, can be pushed through the recording opening 8 along the system axis 4 and, in doing so, the patient can be scanned.

The computed tomography scanner is controlled by a control and computational unit 9, which is connected to the scanning system via a control and data line 10 and also controls the feed of the patient couch 6. The detector output data recorded by the detector array 3 when the patient 7 is scanned are transferred to the control and computational unit 9 via the control and data line 10 and are reconstructed there to form an image, and therefore a slice image or volume display of a region of interest in the patient 7 can be output on a monitor 11 of the control and computational unit 9.

FIG. 2 shows a cross section of a very schematic illustration of a section of the proposed detector array 3. The basic design of the detector array corresponds to that of already known detector arrays with other conversion materials. Such a detector array has individual detector elements 20, which are arranged next to one another in columns, and possibly rows. Each detector element 20 comprises a layer 21 made of the conversion material, which layer is provided with electrode contacts 22 in the case of a direct convertor. If a voltage is applied to these electrode contacts, a suitable electronic circuit 23 then detects and counts the charge pulses generated by incident X-ray or gamma ray quanta. The conversion material of the layer 21 in this example is formed by the element combination TlI (thallium iodide). By selecting the second chemical element with a K-edge in the lower energetic range of ≦30 keV, the conversion material does not have any K-edges in the energetic region of the X-ray radiation. This can avoid fluorescence crosstalk and the dose loss connected therewith.

Such a direct convertor with the proposed conversion material is particularly advantageous because this makes it possible to design significantly smaller detection surfaces or pixels than previously seemed realistic. This firstly has the direct advantage of the higher spatial resolution and the very much improved dose usage connected therewith, for example in CT reconstruction. Furthermore, this allows the implementation of pixel sizes, which are, for example, 25, 36 or 64 times smaller than the pixel sizes of 1 mm², previously used in computed tomography. This quite significantly reduces the maximum X-ray intensity. Thus, the maximum flow can be measured more precisely, or possibly at all, as a result of the maximum count rate per pixel reduced by a factor of 2 to 4.

In summary, using such a detector array can improve the resolution, dose usage, accuracy of the energy measurement and high-flow capability in, for example, CT equipment.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An X-ray or gamma ray detector array comprising:
at least one row of detector elements including at least one layer made of an X-ray or gamma radiation absorbing conversion material, the conversion material being at least in part composed of a chemical element with an atomic number ≧72.

2. The X-ray or gamma ray detector array as claimed in claim 1, wherein the conversion material is composed of a combination of the chemical element with an atomic number ≧72, with one or more additional chemical elements having an atomic number ≦53.

3. The X-ray or gamma ray detector array as claimed in claim 2, wherein the one or more additional chemical elements has an atomic number ≦16.

4. The X-ray or gamma ray detector array as claimed in claim 2, wherein the conversion material is composed of a combination of at least one of the chemical elements W, Tl, Hg, Pb or Bi with at least one of the chemical elements O, S, Se, Te or I.

5. The X-ray or gamma ray detector array as claimed in claim 1, wherein the conversion material is a semiconductor material for direct conversion of the X-ray or gamma radiation into electrical signals.

6. The X-ray or gamma ray detector array as claimed in claim 1, wherein the conversion material is a scintillator material.

7. The X-ray or gamma ray detector array as claimed in claim 1, wherein the X-ray or gamma ray detector array is designed as a counting detector array.

8. The X-ray or gamma ray detector array as claimed in claim 1, wherein the detector elements have a detection surface with dimensions of ≦250 μm in both dimensions.

9. A computed tomography scanner for generating a CT image of an object, comprising:
at least one X-ray source; and
one X-ray detector array as claimed in claim 1, arranged on a rotating frame.

10. The X-ray or gamma ray detector array as claimed in claim 1, wherein the conversion material is at least in part composed of a chemical element with an atomic number >82.

11. The X-ray or gamma ray detector array as claimed in claim 10, wherein the conversion material is composed of a combination of the chemical element with an atomic number >82, with one or more additional chemical elements having an atomic number ≦53.

12. The X-ray or gamma ray detector array as claimed in claim 11, wherein the one or more additional chemical elements has an atomic number ≦16.

13. The X-ray or gamma ray detector array as claimed in claim 11, wherein the conversion material is composed of a combination of at least one of the chemical elements W, Tl, Hg, Pb or Bi with at least one of the chemical elements O, S, Se, Te or I.

14. A computed tomography scanner for generating a CT image of an object, comprising:
at least one X-ray source; and
one X-ray detector array as claimed in claim 2, arranged on a rotating frame.

15. A computed tomography scanner for generating a CT image of an object, comprising:
at least one X-ray source; and
one X-ray detector array as claimed in claim 10, arranged on a rotating frame.
